(19) United States
(12) Patent Application Publication
(10) Pub. No.: US 2025/0336657 A1
(43) Pub. Date: Oct. 30, 2025

(54) Calibration Method, Analysis Method, Controller, and Analyzer

(71) Applicants: SHIMADZU CORPORATION, Kyoto-shi (JP); OSAKA INSTITUTE OF PUBLIC HEALTH, Osaka-shi (JP)

(72) Inventors: Koichi KOJIMA, Kyoto-shi (JP); Yuki WAKABAYASHI, Osaka-shi (JP); Shunya NISHIJIMA, Osaka-shi (JP); Junko SAKATA, Osaka-shi (JP)

(21) Appl. No.: 18/880,115

(22) PCT Filed: Jun. 29, 2023
(86) PCT No.: PCT/JP2023/024120

§ 371 Date: Dec. 30, 2024

(30) Foreign Application Priority Data
Jun. 30, 2022 (JP) 2022-106330

(51) Int. Cl. H01J 49/00

(52) U.S. Cl. CPC H01J 49/0009

(57) ABSTRACT

A method for calibrating a mass spectrum in mass spectrometry of a microorganism sample containing a target substance that is a molecule to be analyzed, including (S31) obtaining a mass spectrum of the microorganism sample to which one or more types of molecules having a smaller estimated theoretical m/z than the target substance are added as a first standard substance; (S32) setting, as a second standard substance, one or more types of molecules derived from the microorganism sample and having a larger estimated theoretical m/z than the target substance; and (S33) calibrating the mass spectrum based on an actual m/z of the first standard substance corresponding to peaks of the mass spectrum and an actual m/z of the second standard substance corresponding to peaks of the mass spectrum, and the theoretical m/z of the first standard substance and the theoretical m/z of the second standard substance.

### TECHNICAL FIELD

The present invention relates to a calibration method, an analysis method, a controller, and an analyzer, and more particularly, relates to a calibration method, an analysis method, a controller, and an analyzer for mass spectrometry.

### BACKGROUND ART

When a target substance that is a molecule to be analyzed is ionized to analyze the mass by mass spectrometry, it is required that the mass of the target substance should be measured with high accuracy. For example, when a polymer having a molecular weight of several thousand Da is to be measured, it is required in some cases to distinguish a difference in the mass value of several Da or less. Accordingly, for increasing the measurement accuracy, the mass of a standard substance that can be calculated for a theoretical value of the mass based on the composition is actually measured, and based on a difference between the actual value and the theoretical value of the mass of the standard substance, a mass spectrometer is calibrated.

### CITATION LIST

### Patent Literature

PTL 1: Japanese Patent Laying-Open No. 2019-90654

### SUMMARY OF INVENTION

### Technical Problem

When a microorganism is subjected to mass spectrometry, a known molecule prepared from E. coli is generally used as a standard substance for calibrating a mass spectrometer. Even a standard substance generally used and having the lowest molecular weight has, however, a mass-to-charge ratio (m/z) of about 4,300, and hence, when a smaller molecule is a target substance, the method is extrapolative, and hence accuracy is reduced.

PTL 1 (Japanese Patent Laying-Open No. 2019-90654) discloses, as a countermeasure, a calibration method using, as a standard substance, aspartate-1-decarboxylase (m/z: about 2,800) contained in an E. coli-derived sample. This method however also has issues: a microorganism other than E. coli does not always contain aspartate-1-decarboxylase, the molecular weight is not always the same, and the protein is not always produced.

Accordingly, in mass spectrometry of a microorganism, particularly when a molecule having a low molecular weight is a target substance, there has been a need for a novel calibration method.

The present disclosure has been devised for solving this problem, and an object thereof is to improve analysis accuracy by improving mass accuracy in a mass spectrum.

### Solution to Problem

A calibration method according to a first aspect of the present disclosure is a calibration method for a mass spectrum in mass spectrometry of a microorganism sample containing a target substance that is a molecule to be analyzed, and includes obtaining a mass spectrum of the microorganism sample to which one or more types of molecules having a smaller estimated theoretical m/z than the target substance are added as a first standard substance; setting, as a second standard substance, one or more types of molecules derived from the microorganism sample and having a larger estimated theoretical m/z than the target substance; and calibrating the mass spectrum based on an actual m/z of the first standard substance and an actual m/z of the second standard substance corresponding to peaks of the mass spectrum, and the theoretical m/z of the first standard substance and the theoretical m/z of the second standard substance.

A controller according to a second aspect of the present disclosure is a controller that executes calibration of a mass spectrum in mass spectrometry of a microorganism sample containing a target substance that is a molecule to be analyzed, and includes a memory, and a processor. The memory stores a theoretical m/z of a first standard substance that is one or more types of molecules having a smaller estimated theoretical m/z than the target substance. The processor is configured to obtain a mass spectrum of the microorganism sample to which the first standard substance is added. The processor is configured to set, as a second standard substance, one or more types of molecules derived from the microorganism sample and having a larger estimated theoretical m/z than the target substance. The processor is configured to calibrate the mass spectrum based on an actual m/z of the first standard substance and an actual m/z of the second standard substance corresponding to peaks of the mass spectrum, and the theoretical m/z of the first standard substance and the second standard substance.

An analyzer according to a third aspect of the present disclosure is an analyzer for performing mass spectrometry on a microorganism sample containing a target substance that is a molecule to be analyzed, and includes a measurement part, a memory, and a processor. The measurement part obtains measurement data of the microorganism sample. The memory stores a theoretical m/z of a first standard substance that is one or more types of molecules having a smaller estimated theoretical m/z than the target substance. The processor is configured to obtain, based on the measurement data, a mass spectrum of the microorganism sample to which the first standard substance is added. The processor is configured to set, as a second standard substance, one or more types of molecules derived from the microorganism sample and having a larger estimated theoretical m/z than the target substance. The processor is configured to calibrate the mass spectrum based on an actual m/z of the first standard substance and an actual m/z of the second standard substance corresponding to peaks of the mass spectrum, and the theoretical m/z of the first standard substance and the second standard substance.

### Advantageous Effects of Invention

According to a calibration method of the present disclosure, by using a first standard substance having a smaller theoretical m/z than a target substance added, and a second standard substance derived from a microorganism sample and having a larger theoretical m/z than the target substance, a mass spectrum can be calibrated in such a manner as to reduce a difference between the theoretical m/z and an actual m/z in these standard substances. In this manner, even when the target substance has a low molecular weight, a mass spectrum in a range corresponding to the molecular weight can be suitably calibrated. Therefore, analysis accuracy can be improved by improving mass accuracy in the mass spectrum.

### BRIEF DESCRIPTION OF DRAWINGS

FIG. 1 is a schematic diagram illustrating the configuration of an analyzer according to a present embodiment.

FIG. 2 is a flowchart illustrating an analysis method according to the present embodiment.

FIG. 3 is a flowchart illustrating a calibration method according to the present embodiment.

FIG. 4 is a diagram illustrating an example of a mass spectrum obtained before performing the calibration method of the present embodiment.

FIG. 5 is a table showing an error in the m/z of a target substance indicated by the mass spectrum of FIG. 4.

FIG. 6 is a diagram illustrating an example of a mass spectrum obtained after subjecting the mass spectrum of FIG. 4 to the calibration method of the present embodiment.

FIG. 7 is a table showing an error in the m/z of the target substance indicated by the mass spectrum of FIG. 6.

### DESCRIPTION OF EMBODIMENTS

Now, an embodiment of the present invention will be described in detail with reference to the accompanying drawings. It is noted that the same or corresponding components are referred to with the same reference signs in the drawings to basically avoid redundant description.

### 1. CONFIGURATION OF ANALYZER

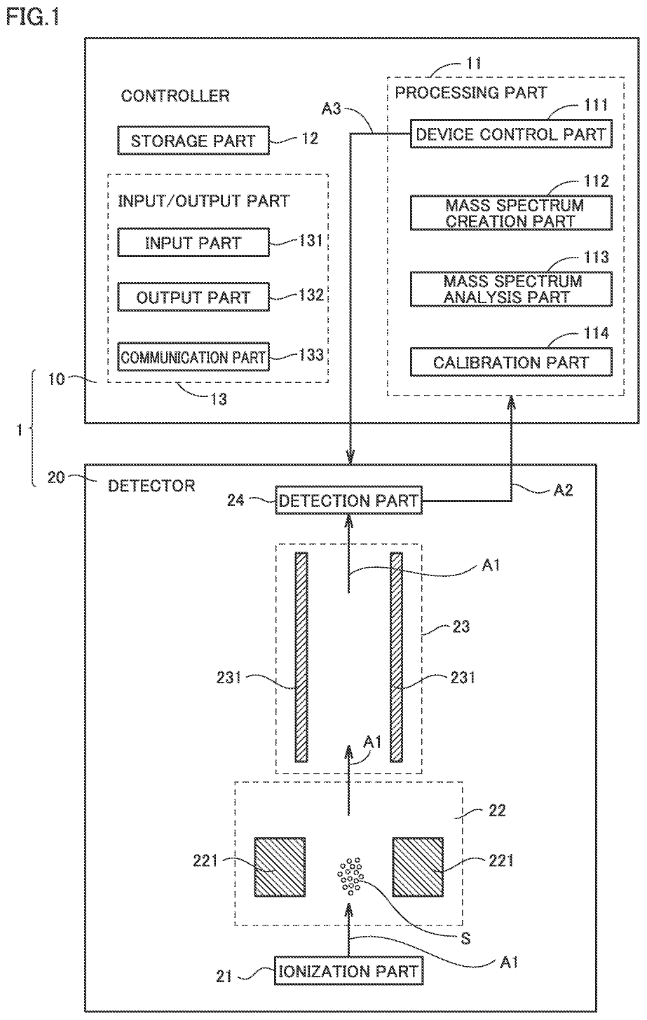

FIG. 1 is a schematic diagram illustrating the configuration of an analyzer 1 according to an embodiment of the present invention. Analyzer 1 is a mass spectrometer for performing mass spectrometry of a substance contained in a sample, and is, for example, MALDI-TOF MS (Matrix-Assisted Laser Desorption/Ionization Time-of-Flight Mass Spectrometer).

In the present embodiment, the sample is an organism sample that is a sample derived from an organism. In one example, the sample is a sample derived from a microorganism. The sample contains a target substance that is a molecule to be analyzed, and a standard substance (calibrant) that is a molecule used in calibration of a mass spectrum. In the present embodiment, analysis with analyzer 1 includes detecting a peak of the mass spectrum, and measuring the m/z of a specific or nonspecific substance contained in the sample. In one example, the substance is a protein. The analysis with analyzer 1 may include discriminating, based on the m/z corresponding to a peak of the mass spectrum (hereinafter, also referred to as the “actual m/z”), whether or not the specific substance is contained in the sample, calculating the concentration of the specific substance in the sample, and identifying an organism contained in the sample.

Referring to FIG. 1, analyzer 1 includes a controller 10 and a detector 20.

Detector 20 ionizes, with a high voltage, a substance (protein) contained in a sample, and detects the resultant ion S after separation in accordance with time of flight correlated with an m/z. Detector 20 includes an ionization part 21, an ion acceleration part 22, a mass separation part 23, and a detection part 24. In FIG. 1, the movement of the ion S in detector 20 is schematically illustrated with an arrow A1.

Ionization part 21 ionizes the substance contained in the sample by matrix-assisted laser desorption/ionization (MALDI) method. As the ionization method, not only MALDI method but also any soft ionization method such as electrospray ionization (ESI) method can be employed. In the ionization performed by ESI method, a configuration in which analyzer 1 further includes a liquid chromatograph for ionizing, with ionization part 21, a substance that is contained in the sample, and has been separated with the liquid chromatograph is preferred because high separability can be thus obtained.

Ionization part 21 includes a sample plate holder (not shown) for holding a sample plate, and an ion source including a laser device (not shown) for irradiating the sample plate with a laser beam. After placing a sample on the sample plate, a matrix is added to the sample, and the resultant sample is dried. Thereafter, the sample plate is set on the sample plate holder disposed in a vacuum container of ionization part 21. The type of the matrix is not especially limited, and from the viewpoint of efficiently ionizing a protein sample, sinapinic acid, α-cyano-4-hydroxycinnamic acid (CHCA), or the like is preferably used.

Ionization part 21 depressurizes the vacuum container in which the sample plate has been set, and then successively irradiates each sample on the sample plate with a laser beam for ionization. The type of the laser device for emitting the laser beam is not especially limited as long as it can oscillate light absorbed by the selected matrix, and for example, when the matrix contains sinapinic acid or CHCA, N2 laser (wavelength: 337 nm) or the like can be suitably used. The ion S having been ionized by ionization part 21 is extracted from an electric field formed by an extraction electrode or the like not shown, and is introduced into ion acceleration part 22.

Ion acceleration part 22 includes an accelerating electrode 221, and accelerates the ion S having been introduced thereinto. The flow of the accelerated ion S is appropriately converged by an ion lens not shown to be introduced into mass separation part 23.

Mass separation part 23 includes a flight tube 231, and separates ions S in accordance with a difference in time of flight spent by the respective ions S flying inside flight tube 231. Although FIG. 1 illustrates linear flight tube 231, a reflectron flight tube, a multi-turn flight tube or the like may be used. The method of mass spectrometry is not especially limited as long as ions S contained in a sample can be separated and detected.

Detection part 24 includes an ion detector such as a multi-channel plate, detects the ion S separated by mass separation part 23, and outputs a detected signal with an intensity according to the number of ions having entered detection part 24. The detected signal output from detection part 24 is input to a processing part 11 of controller 10. In FIG. 1, a flow of the detected signal of the ions S from detection part 24 of detector 20 is schematically illustrated with an arrow A2.

Controller 10 includes processing part 11, a storage part 12, and an input/output part 13. Controller 10 corresponds to one example of a “controller” according to the present disclosure.

Processing part 11 is configured by including a processor such as a CPU, and functions as a main part in an operation for controlling analyzer 1. Processing part 11 performs various processing by executing a program stored in storage part 12 and the like. Processing part 11 corresponds to an example of a “processor” according to the present disclosure.

Processing part 11 includes a device control part 111, a mass spectrum creation part 112, a mass spectrum analysis part 113, and a calibration part 114.

Device control part 111 controls the operation of detector 20 based on data related to analysis conditions input from an input part 131 described below. In FIG. 1, the control of detector 20 by device control part 111 is schematically illustrated with an arrow A3.

Mass spectrum creation part 112 converts the time of flight into an m/z value based on measurement data including the amount of ions detected by detection part 24, and the time of flight of the ions, and creates a mass spectrum indicating the amount detected corresponding to each m/z value.

Mass spectrum analysis part 113 detects, in the mass spectrum, a peak of the mass spectrum. It calculates the m/z corresponding to the detected peak. Mass spectrum analysis part 113 discriminates, based on protein database, a substance corresponding to the actual m/z indicated by the peak of the mass spectrum. In other words, mass spectrum analysis part 113 can calculate an actual m/z of a specific or nonspecific substance contained in the sample. Mass spectrum analysis part 113 may further discriminate, based on the actual m/z, whether or not the specific substance is contained in the sample (component identification in the sample), calculate the concentration of the specific substance in the sample, or identify an organism contained in the sample. More generally, mass spectrum analysis part 113 may perform structural analysis of a substance contained in the sample.

Calibration part 114 calibrates the mass spectrum based on the actual m/z and a theoretical m/z of a standard substance. The theoretical m/z is a value also referred to as a calculated value, a theoretical value, or a theoretical m/z value in general, and is a theoretical mass-to-charge ratio calculated in consideration of the molecular weight, and the numbers of ions and charges added. The calibration in the mass spectrometry means that the actual m/z of the standard substance is corrected to be closer to the theoretical m/z, and the resultant correction is applied to the entire spectrum. The calibration processing will be described in detail below.

Storage part 12 includes a nonvolatile storage medium. Storage part 12 stores the theoretical m/z value, the mass spectrum created by mass spectrum creation part 112, the measurement data output from detector 20, the program used for executing processing by processing part 11, and the like. Storage part 12 corresponds to an example of a “memory” according to the present disclosure.

Input/output part 13 is an interface for inputting/outputting information between analyzer 1 and the outside. Input/output part 13 includes an input part 131, an output part 132, and a communication part 133.

Input part 131 is configured by including an input device such as a mouse, a keyboard, various buttons and/or a touch panel. Input part 131 receives, from a user, information necessary for control of the operation of detector 20, and information necessary for processing performed by processing part 11.

Output part 132 is configured by including a display device such as a liquid crystal monitor, a printer, and the like. Output part 132 displays, in a display device, information on the measurement by detector 20, and results of the processing by processing part 11, or prints these on a print media.

Communication part 133 is configured by including a communication device capable of communication through wireless or wired connection such as Internet. Communication part 133 receives data necessary for processing by processing part 11, transmits data having been processed by processing part 11, such as discrimination results, and appropriately receives/transmits necessary data.

A part or the whole of the function of controller 10 described above may be disposed in a computer, a server, or the like physically separated from detector 20.

### 2. CONVENTIONAL CALIBRATION METHOD

In recent years, a method for identifying a microorganism by mass spectrometry using MALDI method (MALDI-MS) has been rapidly spread. This is because

MALDI-MS does not require any technical skill as compared with a culture method that is a conventional identification method, and can be rapidly performed at low cost. Furthermore, in the conventional identification method, various tests are performed based on some “estimation” about a microorganism such as the name of the genus, and therefore, it is impossible to perform a test, namely, make an identification attempt, unless the “estimation” cannot be made. In contrast, the microorganism identification method by mass spectrometry is an epochal method in the sense that identification can be attempted even without making “estimation” as long as a bacterial body sufficient for preparing a sample for mass spectrometry can be prepared. In other words, in MALDI-MS, identification of a microorganism can be easily attempted as compared with the conventional identification method.

On the other hand, in the microorganism identification by MALDI-MS, a bacterial body is used as a sample without substantially purifying it, and hence, as compared with usual MALDI-MS performed with a protein purified, the amount of the sample, and the number of types of substances contained in the sample are large.

Therefore, there may arise problems that there are so many peaks that the resultant mass spectrum becomes complicated, and that an error of an m/z is increased because of, for example, a rise of the sample. This error can cause a serious problem particularly in analysis for determining from which molecule of a microorganism one peak of a mass spectrum is derived.

In order to reduce such an error in mass spectrometry, mass calibration is an extremely important operation. In mass spectrometry, the accuracy of an m/z in a mass spectrum can be ensured by mass calibration. As a result, identification of a component, identification of a microorganism, and structural analysis utilizing a mass spectrum described above can be suitably executed.

As is well known, the mass calibration method is roughly divided into an internal standard method and an external standard method (see PTL 1 and the like). In the internal standard method, a standard substance is mixed with a sample containing a target substance to be subjected to measurement. In other words, the target substance and the standard substance are simultaneously measured under the same conditions. In a mass spectrum obtained as a result, a m/z value of the mass spectrum is calibrated based on a difference between a theoretical m/z and an actual m/z of the standard substance, and an actual m/z value of the target substance is also calibrated.

On the other hand, in the external standard method, a sample containing a target substance and a sample for calibration containing a standard substance (hereinafter referred to as the “calibration sample”) are not mixed but respectively subjected to measurement. First, in a mass spectrum of the calibration sample, information on a difference between a theoretical m/z and an actual m/z of the standard substance is obtained. Next, the information is used to calibrate a mass spectrum of the sample containing the target substance, and an actual m/z value of the target substance is calibrated. In this external standard method, it is difficult to correct for influence of variation in measurement conditions between the measurement of the sample containing the target substance and the measurement of the calibration sample containing the standard substance. Therefore, more accurate mass calibration is performed generally by the internal standard method than by the external standard method.

Calibration in executing mass spectrometry is extremely important as described above, and application of the internal standard calibration method is desired if possible. In reality, however, the external standard calibration is generally performed because of various technical restriction, cost constraints and the like. On the other hand, when high accuracy is required, various solutions have been searched for within these restrictions and constraints.

When mass spectrometry is performed on a microorganism, calibration is generally performed by the external standard method using, as a standard substance, a substance prepared from E. coli that is well studied and known. The m/z of an E. coli-derived standard substance having the lowest molecular weight (one of ribosome proteins) is, however, about 4,300, and when the target substance is a smaller molecule, the method is extrapolative, and hence the accuracy is liable to be reduced.

As a countermeasure, in the measurement of an E. coli-derived sample, a method in which aspartate-1-decarboxylase (m/z: about 2,800) is used as a novel E. coli-derived standard substance can be employed (PTL 1). However, a microorganism other than E. coli does not always contain aspartate-1-decarboxylase, the amino acid sequence is not always the same, and the protein is not always produced.

As another measure, a method in which a mass spectrum is measured with a standard substance having a low molecular weight and a standard substance having a high molecular weight added to a sample to be analyzed may be employed. When these standard substances are simply added, however, there may arise a problem wherein a multivalent ion having a high molecular weight appears in the range of the m/z of the measurement target in many cases, and hence a mass spectrum of the target substance cannot be obtained.

Considering these problems, in a mass spectrometer according to the present embodiment, first, one or more types of molecules having a known molecular weight and having a lower molecular weight than a target substance are added to a sample as a first standard substance, and a mass spectrum of the resultant sample is measured. Next, one or more types of molecules derived from the sample and having a higher molecular weight than the target substance is set as a second standard substance. Then, the mass spectrum is calibrated in such a manner that an actual m/z corresponding to the first standard substance indicated by the mass spectrum of the sample, and an actual m/z corresponding to the second standard substance respectively correspond to a theoretical m/z corresponding to the first standard substance and a theoretical m/z corresponding to the second standard substance. The m/z of the mass spectrum thus calibrated is improved in the mass accuracy, and the accuracy of analysis using such a mass spectrum is also improved. Accordingly, in the mass spectrometer according to the present embodiment, the mass accuracy can be improved by such a simple calibration method, and hence analysis accuracy can be improved.

### 3. ANALYSIS METHOD OF PRESENT EMBODIMENT

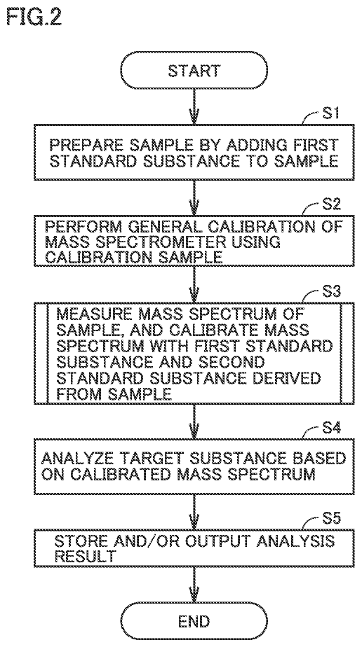

FIG. 2 is a flowchart illustrating an analysis method according to the present embodiment. Steps illustrated in FIG. 2 are executed by analyzer 1 and a user. In the drawing, “S” is used as an abbreviation of “STEP”.

In S1, a user prepares a sample by adding a first standard substance to the sample. The first standard substance is one or more types of molecules having a smaller estimated theoretical m/z than a target substance. For example, when the purpose of analysis performed with analyzer 1 is identification of a microorganism contained in the sample, the m/z of the substance referred to in the identification of the microorganism corresponding to the target substance in this case is generally about 3,000 to 15,000. Therefore, the m/z of the first standard substance used in this case is preferably less than about 3,000, and more preferably less than about 1,500. The first standard substance is, for example, a protein or peptide. A more specific example of the first standard substance is angiotensin II (molecular weight: 1,046.2) and/or angiotensin I (molecular weight: 1296.5). The first standard substance is, however, not limited to this, but may contain, for example, at least one of Bradykinin Fragment 1-7 (human) (molecular weight: about 757.4), P14R (synthetic peptide) (molecular weight: about 1533.9), ACTH fragment 18-39 (human) (molecular weight: about 2465.2), and oxidized insulin B-chain (bovine) (molecular weight: about 3494.7).

In one realization example, the theoretical m/z of the first standard substance is estimated by obtaining the molecular weight based on the amino acid sequence of the first standard substance, and calculating the theoretical m/z based on the molecular weight. The theoretical m/z is simply estimated, for example, by adding 1.08, that is, the mass of a hydrogen atom, to the molecular weight. When there is database including the theoretical m/z or the molecular weight of the first standard substance, the theoretical m/z or the molecular weight of the first standard substance may be obtained from the database.

In S2, the user performs general calibration of a mass spectrometer in analyzer 1 by using a calibration sample. The calibration sample is generally a sample containing a plurality of standard substances, and a product commercially available as a sample for calibration of a mass spectrometer may be used. The calibration sample may or may not contain a first standard substance and a second standard substance.

The calibration using the calibration sample performed in S2 is calibration generally performed before measuring a target substance in a mass spectrometer, but is not always necessary when analyzer 1 is suitably adjusted and managed.

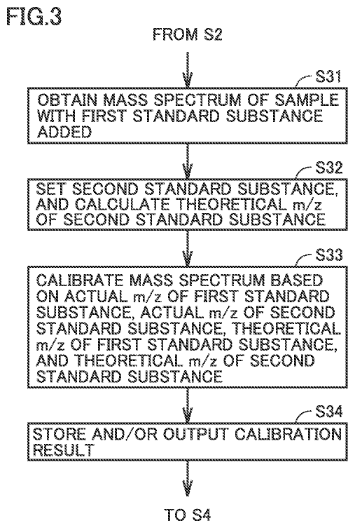

In S3, the user measures, in analyzer 1, a mass spectrum of the sample prepared in S1, and calibrates the mass spectrum with the first standard substance, and a second standard substance derived from the sample. The second standard substance is one or more types of molecules derived from a microorganism contained in the sample and having a larger estimated theoretical m/z than the target substance. The second standard substance is, for example, a protein. Details of the processing performed in S3 will be described in detail referring to FIG. 3.

In S4, the user analyzes, with analyzer 1, the target substance based on the calibrated mass spectrum.

In S5, the user causes analyzer 1 to store and/or display the analysis result obtained in S4. In this manner, the user can easily store and/or recognize the analysis result.

### 4. CALIBRATION METHOD OF PRESENT EMBODIMENT

FIG. 3 is a flowchart illustrating a calibration method according to the present embodiment. S31 to S34 illustrated in FIG. 3 are processing corresponding to S3 of FIG. 2. The respective steps illustrated in FIG. 3 are executed in processing part 11 of analyzer 1.

In S31, processing part 11 obtains the mass spectrum of the sample to which the first standard substance has been added. The user and/or processing part 11 may set, in S31, conditions for measuring a mass spectrum before starting the measurement of the mass spectrum. For example, a mass range for measuring the mass spectrum is set to include the m/z of the first standard substance. Other conditions for measuring the mass spectrum are appropriately set so that a clear spectrum can be obtained.

In S32, processing part 11 sets a second standard substance. As the second standard substance, for example, a ribosome protein is set. A ribosome protein of a microorganism is clearly observed by MALDI in many cases, the amino acid sequence thereof can be checked in public database such as UniProt in many cases, and it is easy to calculate, based on the amino acid sequence, the molecular weight, and further the theoretical m/z. Therefore, when a microorganism contained in the sample is known or predicted, it is useful to set a ribosome protein of the microorganism as the second standard substance.

The second standard substance is set, for example, with input part 131 by the user. The setting by the user is performed through a process wherein, for example, when the mass spectrum is displayed in output part 132 and the user selects a prescribed peak in the mass spectrum with input part 131, a molecule corresponding to the peak is set as the second standard substance. Alternatively, the setting by the user may be performed, for example, by causing the user to input, with input part 131, information on a substance to be used as the second standard substance, or to select it from choices.

The setting of the second standard substance in S32 includes estimation of a theoretical m/z of the second standard substance. In one realization example, the theoretical m/z of the second standard substance is estimated by obtaining the molecular weight based on the amino acid sequence of the second standard substance, and calculating the theoretical m/z based on the molecular weight. The theoretical m/z is simply estimated by, for example, multiplying the molecular weight by 1.08, that is, the mass of a hydrogen atom. When there is database including the theoretical m/z or the molecular weight of the second standard substance, the theoretical m/z or the molecular weight of the second standard substance may be obtained from the database.

In S33, processing part 11 calibrates the mass spectrum based on the actual m/z of the first standard substance and the actual m/z of the second standard substance corresponding to the peaks of the mass spectrum, and the theoretical m/z of the first standard substance and the second standard substance.

First, processing part 11 detects the actual m/z of the first standard substance and the actual m/z of the second standard substance from the mass spectrum. For example, processing part 11 detects, in the mass spectrum, the actual m/z of the first standard substance by determining that an actual m/z of a peak included within a prescribed error margin from the theoretical m/z of the first standard substance is the actual m/z of the first standard substance. The error margin is, for example, previously set in numerical values. The detection of the actual m/z of the first standard substance is not limited to this, and a person skilled in the art may determine, through observation of the mass spectrum, that an actual m/z of a peak determined to be included within a prescribed error margin from the theoretical m/z of the first standard substance is the actual m/z of the first standard substance. Similarly, processing part 11 detects the actual m/z of the second standard substance.

Next, processing part 11 accepts selection of one or more molecules to be used for the calibration from molecules corresponding to the first standard substance. This selection is performed, for example, by the user selecting, with input part 131, a peak corresponding to the molecule on the mass spectrum. Similarly, processing part 11 accepts selection of one or more molecules to be used for the calibration from molecules corresponding to the second standard substance. The thus selected one or more molecules corresponding to the first standard substance are hereinafter referred to as the “first molecule”. The thus selected one or more molecules corresponding to the second standard substance are hereinafter referred to as the “second molecule”.

Processing part 11 calibrates the mass spectrum based on the actual m/z of the first molecule, the actual m/z of the second molecule, the theoretical m/z of the first molecule and the theoretical m/z of the second molecule. The calibration of the mass spectrum is, for example, performed as follows.

When each of the first molecule and the second molecule includes one molecule, the calibration is performed in such a manner that the actual m/z of the first molecule can be the theoretical m/z of the first molecule, and that the actual m/z of the second molecule can be the theoretical m/z of the second molecule. Then, in accordance with this calibration, the m/z on the mass spectrum is linearly calibrated.

When at least one of the first molecule and the second molecule includes a plurality of molecules, the calibration is performed in such a manner that the actual m/z of each molecule can become closer to the theoretical m/z of the corresponding molecule. As one example, under conditions that the m/z on the mass spectrum is to be linearly calibrated, the mass spectrum is calibrated in such a manner as to minimize a sum of squares, of all the molecules, of an error between the actual m/z and the theoretical m/z of each of these molecules. Specifically, the mass spectrum is calibrated in such a manner as to reduce a sum of a square of a difference between the actual m/z and the theoretical m/z of the first standard substance and a square of a difference between the actual m/z and the theoretical m/z of the second standard substance. As another example, the calibration is performed in such a manner that the actual m/z of each of these molecules can be the theoretical m/z thereof, and the actual m/z values between these molecules are also linearly calibrated.

The method for calibrating a mass spectrum based on a first standard substance and a second standard substance is not limited to those described above, and any method may be employed as long as the calibration is performed in such a manner that the actual m/z of the first standard substance can become closer to the theoretical m/z of the first standard substance, and that the actual m/z of the second standard substance can become closer to the theoretical m/z of the second standard substance, and the resultant calibration is applied to the entire spectrum. In other words, the mass spectrum may be calibrated in such a manner as to reduce both of a difference between the actual m/z and the theoretical m/z of the first standard substance, and a difference between the actual m/z and the theoretical m/z of the second standard substance. For example, any one of, or a combination of a plurality of methods already known as a calibration method using a standard substance may be used.

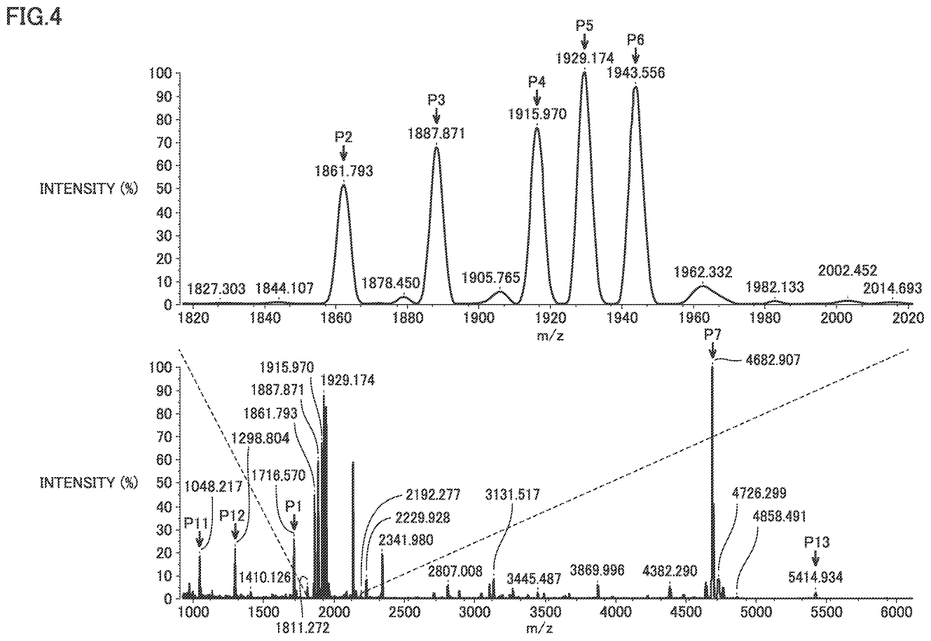

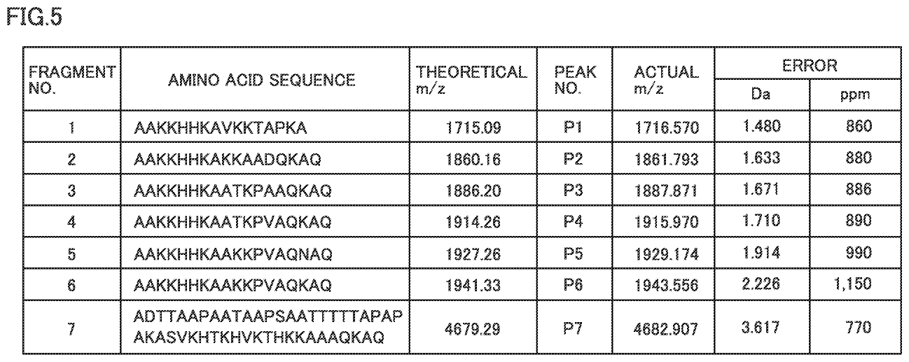

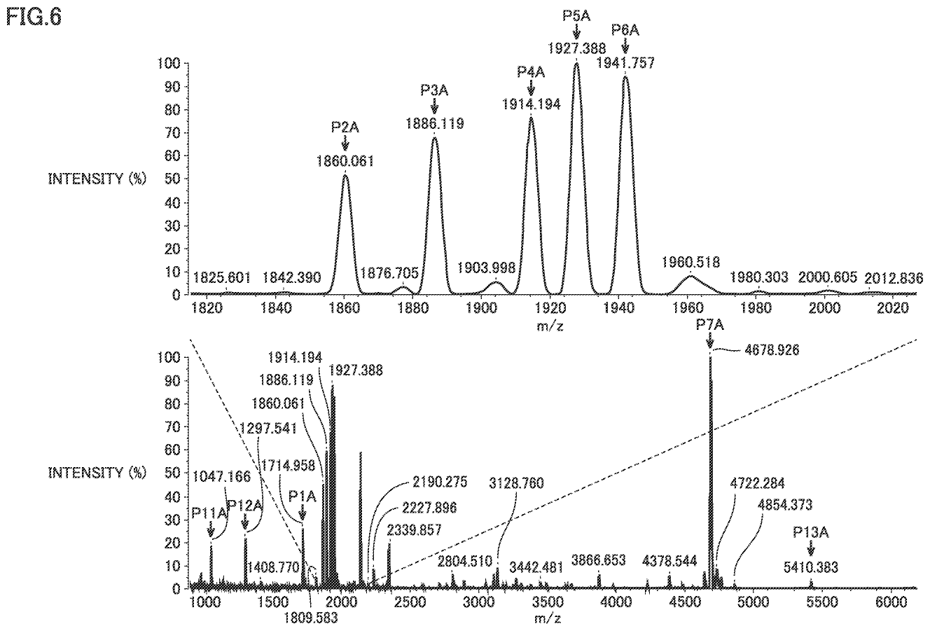

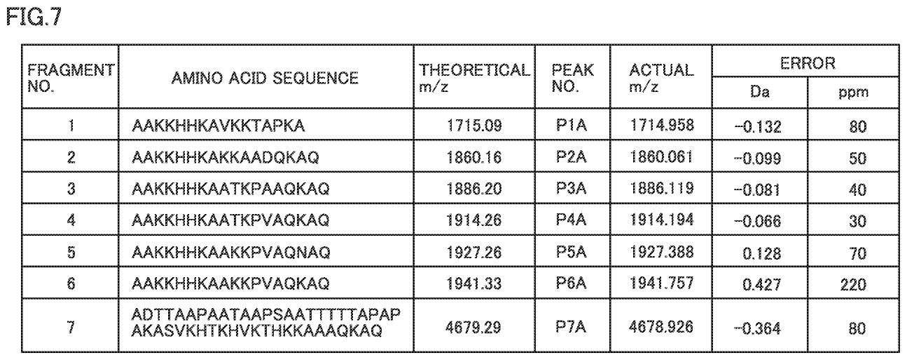

In S34, processing part 11 stores the calibration result in storage part 12, and/or outputs it from output part 132 if necessary. The calibration result includes, for example, at least one of a calibrated mass spectrum, a calibrated actual m/z of the specific substance, and an error value of the calibrated actual m/z of the specific substance from a calibrated theoretical m/z (see, for example, FIG. 6 and FIG. 7 described below). Thus, the user can easily store and/or recognize the calibration result. When the calibration result includes the above-described indices obtained before the calibration, the effects obtained before and after the calibration can be compared with each other (see, for example, FIG. 4 and FIG. 5 described below).

According to the calibration method of the present embodiment illustrated in FIG. 3, a first standard substance having a smaller theoretical m/z than a target substance added, and a second standard substance derived from a biological sample and having a larger theoretical m/z than the target substance are used, and the calibration is performed in such a manner as to reduce a difference between the actual m/z and the theoretical m/z in each of the standard substances. Then, in accordance with the calibration, the actual m/z of the target substance having a theoretical m/z between the theoretical m/z of the first standard substance and the theoretical m/z of the second standard substance is also calibrated. Therefore, the analysis accuracy can be improved by improving the mass accuracy in the mass spectrometer.

According to this calibration method, as compared with an external standard method using, as a standard substance, a substance prepared from a general E. coli, a first standard substance having a smaller theoretical m/z is used, and hence, a smaller m/z range can be suitably calibrated. Therefore, even a target substance having a low molecular weight can be accurately analyzed.

Furthermore, in a general external standard method, it is necessary to set conditions for measurement of a sample according to conditions for measurement of a calibration sample containing a standard substance, but in the calibration method of the present embodiment, there is no such restriction because the internal standard method is employed. Therefore, a burden on a user in setting the measurement conditions is reduced.

Furthermore, in S32, the theoretical m/z of the second standard substance may be estimated based on the actual m/z of the second standard substance previously measured by an internal standard method instead of being calculated based on the amino acid sequence. Specifically, the actual m/z obtained by precedent measurement of the second standard substance by the internal standard method can be used as the theoretical m/z of the second standard substance in the present embodiment. Since an actual m/z measured by the internal standard method is sufficiently accurate in many cases as compared with an actual m/z measured by an external standard method, the theoretical m/z of the second standard substance thus obtained is sufficiently accurate in many cases in the present embodiment. This method is useful if the theoretical m/z cannot be calculated, for example, because the amino acid sequence is unknown. This method is very useful because it is naturally applicable to a molecule other than a protein, and to a molecule having been post-translationally modified.

The calibration method of the present embodiment illustrated in FIG. 3 can be executed when a mass spectrum of a biological sample to which a first standard substance has been added is obtained. Therefore, detector 20 is not essential in analyzer 1, and the calibration method of the present embodiment may be executed based on the mass spectrum obtained from the outside by controller 10.

Furthermore, in the respective steps illustrated in FIG. 2 and FIG. 3, the operations and the setting performed by the user may be appropriately automated to be performed by analyzer 1. For example, the configuration as follows may be employed: When a sample prepared by the user is set in analyzer 1 in S1, and information based on which a theoretical value m/z of the target substance can be estimated, information based on which a theoretical value m/z of the first standard substance can be estimated, and information on which a theoretical value m/z of the second standard substance can be estimated are input by the user to analyzer 1, and then, subsequent steps are automatically performed by analyzer 1 by inputting an instruction to execute the calibration method of the present embodiment.

Furthermore, in the respective processings illustrated in FIG. 2 and FIG. 3, when the same effect can be attained even if the orders of processings are changed, the orders of the processings may be changed, and when the same effect can be attained even if processings are simultaneously performed, the processings may be simultaneously performed.

### 5. EXAMPLES

Next, examples performed for verifying the effect of the present invention will be described.

### 5-1. Setting of Target Substance

In this example, an acid shock protein fragment of Erwinia persicina was used as a target substance. Most of microorganisms belonging to the order Enterobacterales have their respective genes. The amino acid sequences thereof are, however, largely different and diverse among different genera and species, and it is presumed that the acid shock protein is cleaved at a specific sequence to be fragmented, which is still under research and has not been determined. Through research made by the present inventors, it has been revealed that the acid shock protein of the order Enterobacterales is produced by using a sugar-supplemented medium. When an analysis target is diverse and undetermined in this manner, correctness of the hypothesis can be increased by minimizing an error of a value deduced from the hypothesis, which is an error between a theoretical m/z estimated from the amino acid sequence of a fragment and an actual m/z in this case. In other words, it is necessary to obtain, with higher accuracy, the actual m/z of a mass spectrum peak corresponding to a target substance by calibration and the like.

### 5-2. Culture Conditions

In order to produce a larger amount of the acid shock protein, Erwinia persicina was cultured in IFO 804 medium (0.5% of hipolypepton, 0.5% of yeast extract, 0.5% of glucose, 0.1% of magnesium sulfate) overnight (18 hours). 1 mL of a suspension of Erwinia persicina thus proliferated was centrifuged, a supernatant was discarded to obtain a precipitate of the microorganism, the precipitate was further suspended again in purified water, the resultant was centrifuged again, a supernatant was discarded, and thus, a precipitate washed with purified water was obtained. A 1% trifluoroacetic acid solution was added to this precipitate for lysis.

### 5-3. Preparation of Sample

To the thus obtained lysate, angiotensin 1 (molecular weight: 1296.5, m/z of theoretical m/z: 1297.5) and angiotensin 2 (molecular weight: 1046.2, m/z of theoretical m/z: 1047.2) were added to a final concentration of 0.1 pmol/μL as the first standard substance having a theoretical m/z smaller than the theoretical m/z of the target substance, which is one of characteristics of the analysis method of the present embodiment.

The lysate to which the first standard substance had been added, and 10 mg/mL «-cyano-4-hydroxycinnamic acid (CHCA) were mixed in equal amounts, and 1 μL of the resultant mixture was dropped onto a target plate of MALDI, and solidified by drying to be used as a sample of mass spectrum measurement.

### 5-4. Obtaining Mass Spectrum

The sample was set in a mass spectrometer (manufactured by Shimadzu Corporation, MALDI-8020), and the measurement was performed with a measurement range set to m/z of 900 to 15,000 in the positive linear mode to obtain a mass spectrum illustrated in FIG. 4.

FIG. 4 is a diagram illustrating an example of a mass spectrum obtained before performing the calibration method of the present embodiment. In the lower portion of FIG. 4, a mass spectrum in a range of the m/z of about 1,000 to about 6,000 is illustrated. In the upper portion of FIG. 4, an enlarged diagram of a mass spectrum in a range of the m/z of about 1,800 to about 2,000 is illustrated. In FIG. 4, the abscissa indicates the m/z, and the ordinate indicates the ratio of signal intensity. Furthermore, a numerical value shown above each peak indicates the m/z corresponding to the peak. P11 and P12 indicate peaks corresponding to angiotensin 2 and angiotensin 1. P13 indicates a peak corresponding to 50S ribosomal protein L34 of Erwinia persicina used as a second standard substance described below. P1 to P7 indicate peaks corresponding to 7 fragments of an acid shock protein described below.

FIG. 5 is a table showing an error in the m/z of the target substance indicated by the mass spectrum of FIG. 4. FIG. 5 was used for calculating an error in the m/z of the target substance. Through the research made by the present inventors, it was predicted that the acid shock protein of Erwinia persicina would be cleaved into 7 fragments. “FRAGMENT NOS.” 1 to 7 are assigned to these 7 fragments, and amino acid sequences corresponding to the fragments 1 to 7 are shown in the column of “AMINO ACID SEQUENCE”. Theoretical m/zs of the fragments 1 to 7 estimated based on the amino acid sequences are shown in the column of “THEORETICAL m/z”. The peak Nos. P1 to P7 determined to correspond to the fragments 1 to 7 in the mass spectrum of FIG. 4 are shown in the column of “PEAK NO.”. The m/zs corresponding to the peaks P1 to P7 are shown in the column of “ACTUAL m/z”. An error between the actual m/z and the theoretical m/z is shown in the column of “ERROR” in units of Da and ppm.

### 5-5. Calibration based on First Standard Substance and Second Standard Substance

Next, the calibration based on the first standard substance and the second standard substance, which is another characteristic of the analysis method of the present embodiment, was performed. Specifically, for the peak of the actual m/z of 1048.217 determined to correspond to angiotensin 2, 1047.2 that is the theoretical m/z of angiotensin 2 was input as a set value. A set value refers to a target value to which an actual m/z is made to be closer in the calibration. In this example, analyzer 1 was used to calibrate the m/z of the mass spectrum in such a manner that the actual m/z of the standard substance could become the set value in the calibration. Similarly, for the peak of the actual m/z of 1298.804 determined to correspond to angiotensin 1, 1297.5 that is the theoretical m/z thereof was input as a set value.

Then, as a peak of the second standard substance derived from the sample and having a theoretical m/z larger than the theoretical m/z of the target substance, the peak of the actual m/z of 5414.934 determined to correspond to 50S ribosomal protein L34 of Erwinia persicina was selected. Then, for the selected peak, the theoretical m/z thereof of 5,410.39 was input as a set value. Thereafter, analyzer 1 was used to calibrate the mass spectrum based on the set value of the first standard substance and the set value of the second standard substance, and thus, a mass spectrum after the calibration illustrated in FIG. 6 was obtained.

FIG. 6 is a diagram illustrating an example of a mass spectrum obtained after performing the calibration method of the present embodiment. In the same manner as in FIG. 4, in the lower portion of FIG. 6, a mass spectrum in a range of the m/z of about 1,000 to about 6,000 is illustrated. In the upper portion of FIG. 6, an enlarged diagram of a mass spectrum in a range of the m/z of about 1,800 to about 2,000 is illustrated. In FIG. 6, the abscissa indicates the m/z, and the ordinate indicates the ratio of signal intensity. Furthermore, a numerical value shown above each peak indicates the m/z corresponding to the peak. P11A and P12A indicate peaks corresponding to angiotensin 2 and angiotensin 1. P13A indicates a peak corresponding to 50S ribosomal protein L34 of Erwinia persicina used as the second standard substance. P1A to P7A indicate peaks corresponding to the 7 fragments of the acid shock protein.

FIG. 7 is a table showing an error in the m/z of the target substance indicated by the mass spectrum of FIG. 6. FIG. 7 was used for calculating an error in the m/z of the target substance. In the same manner as in FIG. 5, information corresponding to the fragments 1 to 7 of the acid shock protein of Erwinia persicina is shown. Specifically, in the columns of “AMINO ACID SEQUENCE”, “THEORETICAL m/z”, and “PEAK NO.” of FIG. 7, the same values as those shown in FIG. 5 are shown. In the column of “ACTUAL m/z” of FIG. 7, values of the calibrated actual m/zs are shown. Then, an error between the calibrated actual m/z and the theoretical m/z is shown in the column of “ERROR”.

### 5-6. Verification of Effect of Calibration Method of Present Embodiment

The mass spectrum of FIG. 4 and the table of FIG. 5 are obtained before performing the calibration based on the first standard substance and the second standard substance (FIG. 3), and can be regarded as those obtained without applying the calibration method of the present embodiment. In this case, the errors of the actual m/zs from the theoretical m/zs of the predicted fragments 1 to 7 of the acid shock protein were 1.4 Da (860 ppm) or more.

In contrast, the mass spectrum of FIG. 6 and the table of FIG. 7 are obtained after performing the calibration based on the first standard substance and the second standard substance (FIG. 3), and can be regarded as those obtained by applying the calibration method of the present embodiment. In this case, the errors of the actual m/zs from the theoretical m/zs of the predicted fragments 1 to 7 of the acid shock protein were 0.43 Da (220 ppm) or less. In other words, in the calibrated mass spectrum, the error in the m/z of the target substance was largely reduced.

As described above, the calibration method of the present embodiment was executed on the estimated fragments of the acid shock protein of Erwinia persicina to improve the mass accuracy, and thus, the correctness of the estimation could be verified with higher accuracy. In other words, by executing the calibration method of the present embodiment, the analysis of a microorganism molecule by mass spectrometry could be simply and more highly accurately executed.

### Aspects

Those skilled in the art will understand that the above-described plurality of exemplifying embodiments are specific examples of the following aspects.

(Item 1) A calibration method according to one aspect is a method for calibrating a mass spectrum in mass spectrometry of a microorganism sample containing a target substance that is a molecule to be analyzed, and the method includes obtaining a mass spectrum of the microorganism sample to which one or more types of molecules having a smaller estimated theoretical m/z than the target substance are added as a first standard substance; setting, as a second standard substance, one or more types of molecules derived from the microorganism sample and having a larger estimated theoretical m/z than the target substance; and calibrating the mass spectrum based on an actual m/z of the first standard substance corresponding to peaks of the mass spectrum and an actual m/z of the second standard substance corresponding to peaks of the mass spectrum, and the theoretical m/z of the first standard substance and the theoretical m/z of the second standard substance.

In the calibration method according to item 1, by using a first standard substance having a smaller theoretical m/z than a target substance added, and a second standard substance derived from a microorganism sample and having a larger theoretical m/z than the target substance, a mass spectrum can be calibrated in such a manner as to reduce a difference between the theoretical m/z and an actual m/z in these standard substances. Thus, even when the target substance has a low molecular weight, a mass spectrum in a range corresponding to the molecular weight can be suitably calibrated. Therefore, analysis accuracy can be improved by improving mass accuracy in the mass spectrum.

(Item 2) In the calibration method according to item 1, the calibrating includes calibrating the mass spectrum in such a manner as to reduce a sum of a square of a difference between the actual m/z and the theoretical m/z of the first standard substance and a square of a difference of an error between the actual m/z and the theoretical m/z of the second standard substance.

In the calibration method according to item 2, the mass spectrum can be calibrated by simple calculation in such a manner as to make the actual m/z closer to the theoretical m/z in both the first standard substance and the second standard substance. In other words, the range of the m/z corresponding to the target substance in the mass spectrum can be suitably calibrated by simple calculation.

(Item 3) In the calibration method according to item 1 or 2, the first standard substance is a protein or peptide.

In the calibration method according to item 3, the calibration method of item 1 can be executed with one of a protein or peptide having a known molecular weight selected as the first standard substance in accordance with the target substance.

(Item 4) In the calibration method according to any one of items 1 to 3, the first standard substance contains at least one of angiotensin 1, angiotensin 2, Bradykinin Fragment (1-7), P14R, ACTH fragment (18-39), and oxidized insulin B-chain.

In the calibration method according to item 4, the calibration method of item 1 can be executed with at least one of the above-described molecules having a comparatively low molecular weight selected as the first standard substance in accordance with the target substance.

(Item 5) In the calibration method according to any one of items 1 to 4, the second standard substance contains a ribosome protein.

A ribosome protein of a microorganism is clearly observed by MALDI in many cases, the amino acid sequence thereof can be checked in public database such as UniProt in many cases, and it is easy to calculate, based on the amino acid sequence, the molecular weight, and further the theoretical m/z. Therefore, according to the calibration method of item 5, the second standard substance with which the theoretical m/z can be easily calculated owing to these properties of the ribosome protein can be simply set.

(Item 6) In the calibration method according to any one of items 1 to 5, the theoretical m/z of the second standard substance is estimated based on an amino acid sequence of the second standard substance.

According to the calibration method of item 6, the theoretical m/z of the second standard substance can be simply calculated based on the amino acid sequence.

(Item 7) In the calibration method according to any one of items 1 to 5, the theoretical m/z of the second standard substance is estimated based on an actual m/z of the second standard substance having been previously measured by an internal standard method.

In the calibration method of item 7, an actual m/z measured by the internal standard method is sufficiently accurate in many cases, and therefore, even when the theoretical m/z of the second standard substance is set in this manner, the accuracy is sufficient in many cases. This method is particularly useful when the theoretical m/z cannot be calculated, for example, because the amino acid sequence is unknown.

(Item 8) An analysis method further including analyzing a target substance based on a mass spectrum having been calibrated by employing the calibration method according to any one of items 1 to 7.

According to the calibration method of item 8, the target substance can be analyzed by the analysis method having improved analysis accuracy. Accordingly, a more precise analysis result can be obtained.

(Item 9) A controller according to another aspect is a controller that executes calibration of a mass spectrum in mass spectrometry of a microorganism sample containing a target substance that is a molecule to be analyzed, and includes a memory and a processor. The memory stores a theoretical m/z of a first standard substance that is one or more types of molecules having a smaller estimated theoretical m/z than the target substance. The processor is configured to obtain a mass spectrum of the microorganism sample to which the first standard substance is added. The processor is configured to set, as a second standard substance, one or more types of molecules derived from the microorganism sample and having a larger estimated theoretical m/z than the target substance. The processor is configured to calibrate the mass spectrum based on an actual m/z of the first standard substance corresponding to peaks of the mass spectrum and an actual m/z of the second standard substance corresponding to peaks of the mass spectrum, and the theoretical m/z of the first standard substance and the second standard substance.

According to the controller of item 9, by using a first standard substance having a smaller theoretical m/z than a target substance added, and a second standard substance derived from a microorganism sample and having a larger theoretical m/z than the target substance, a mass spectrum can be calibrated in such a manner as to reduce a difference between the theoretical m/z and an actual m/z in these standard substances. Thus, even when the target substance has a low molecular weight, a mass spectrum in a range corresponding to the molecular weight can be suitably calibrated. Therefore, analysis accuracy can be improved by improving mass accuracy.

(Item 10) An analyzer according to another aspect is an analyzer that performs mass spectrometry of a microorganism sample containing a target substance that is a molecule to be analyzed, and includes a detector, a memory, and a processor. The detector obtains measurement data of the microorganism sample. The memory stores a theoretical m/z of a first standard substance that is one or more types of molecules having a smaller estimated theoretical m/z than the target substance. The processor is configured to obtain, based on the measurement data, a mass spectrum of the microorganism sample to which the first standard substance is added. The processor is configured to set, as a second standard substance, one or more types of molecules derived from the microorganism sample and having a larger estimated theoretical m/z than the target substance. The processor is configured to calibrate the mass spectrum based on an actual m/z of the first standard substance and an actual m/z of the second standard substance corresponding to peaks of the mass spectrum, and the theoretical m/z of the first standard substance and the second standard substance.

According to the analyzer of item 9, by using a first standard substance having a smaller theoretical m/z than the target substance added, and a second standard substance derived from a microorganism sample and having a larger theoretical m/z than the target substance, a mass spectrum can be calibrated in such a manner as to reduce a difference between the theoretical m/z and an actual m/z in these standard substances. Thus, even when the target substance has a low molecular weight, a mass spectrum in a range corresponding to the molecular weight can be suitably calibrated. Therefore, analysis accuracy of the analyzer can be improved by improving mass accuracy in the mass spectrum.

It should be recognized that the embodiments disclosed herein are not limiting but illustrative in all points. The scope of the present invention is not limited by the description given above but limited by the scope of appended claims, and is intended to encompass equivalence of the scope of appended claims, and all modifications made within the scope.

### REFERENCE SIGNS LIST

1 Analyzer, 10 Controller, 11 Processing part; 12 Storage part; 13 Input/output part; 18 Fragment; 20 Detector, 21 Ionization part; 22 Ion acceleration part; 23 Mass separation part; 24 Detection part; 111 Device control part; 112 Mass spectrum creation part; 113 Mass spectrum analysis part; 114 Calibration part; 131 Input part; 132 Output part; 133 Communication part; 221 Acceleration electrode; 231 Flight tube; S Ion.

1. A method executed by a computer for calibrating a mass spectrum in mass spectrometry of a microorganism sample containing a target substance that is a molecule to be analyzed, the method comprising:
obtaining a mass spectrum of the microorganism sample to which one or more types of molecules having a smaller theoretical m/z than the target substance are added as a first standard substance;
setting, as a second standard substance, one or more types of molecules derived from the microorganism sample and having a larger theoretical m/z than the target substance; and
calibrating the mass spectrum based on an actual m/z of the first standard substance corresponding to peaks of the mass spectrum and an actual m/z of the second standard substance corresponding to peaks of the mass spectrum, and the theoretical m/z of the first standard substance and the theoretical m/z of the second standard substance.

2. The calibration method according to claim 1, wherein the calibrating includes calibrating the mass spectrum in such a manner as to reduce a sum of a square of a difference between the actual m/z and the theoretical m/z of the first standard substance and a square of a difference between the actual m/z and the theoretical m/z of the second standard substance.

3. The calibration method according to claim 1, wherein the first standard substance is a protein or peptide.

4. The calibration method according to claim 1, wherein the first standard substance contains at least one of angiotensin 1, angiotensin 2, Bradykinin Fragment (1-7), P14R, ACTH fragment (18-39), and oxidized insulin B-chain.

5. The calibration method according to claim 1, wherein the second standard substance contains a ribosome protein.

6. The calibration method according to claim 1, wherein the theoretical m/z of the second standard substance is calculated based on an amino acid sequence of the second standard substance.

7. The calibration method according to claim 1, wherein the theoretical m/z of the second standard substance is an actual m/z of the second standard substance having been previously measured by an internal standard method.

8. An analysis method comprising analyzing the target substance based on a mass spectrum having been calibrated by employing the calibration method according to claim 1.

9. A controller that executes calibration of a mass spectrum in mass spectrometry of a microorganism sample containing a target substance that is a molecule to be analyzed, the controller comprising:
a memory that stores a theoretical m/z of a first standard substance that is one or more types of molecules having a smaller theoretical m/z than the target substance; and
a processor,
wherein the processor is configured to
obtain a mass spectrum of the microorganism sample to which the first standard substance is added;
set, as a second standard substance, one or more types of molecules derived from the microorganism sample and having a larger theoretical m/z than the target substance; and
calibrate the mass spectrum based on an actual m/z of the first standard substance corresponding to peaks of the mass spectrum and an actual m/z of the second standard substance corresponding to peaks of the mass spectrum, and the theoretical m/z of the first standard substance and the theoretical m/z of the second standard substance.

10. An analyzer that performs mass spectrometry of a microorganism sample containing a target substance that is a molecule to be analyzed, comprising:
a detector that obtains measurement data of the microorganism sample; and
the controller according to claim 9.

11. A method executed by a computer for calibrating a mass spectrum in mass spectrometry of a microorganism sample containing a target substance that is a molecule to be analyzed, the method comprising:
obtaining a mass spectrum of the microorganism sample to which one or more types of molecules having a smaller theoretical m/z than the target substance are added as a standard substance; and
calibrating the mass spectrum based on an actual m/z of the standard substance corresponding to peaks of the mass spectrum and the theoretical m/z of the standard substance.

12. The calibration method according to claim 11, wherein the standard substance is a protein or peptide.

13. The calibration method according to claim 11, wherein the standard substance contains at least one of angiotensin 1, angiotensin 2, Bradykinin Fragment (1-7), P14R, ACTH fragment (18-39), and oxidized insulin B-chain.

14. An analysis method comprising analyzing the target substance based on a mass spectrum having been calibrated by employing the calibration method according to claim 11.
